(19) United States
(12) Patent Application Publication
(10) Pub. No.: US 2019/0025271 A1
(43) Pub. Date: Jan. 24, 2019

(54) CHEMICALLY ROBUST MINIATURE GAS SENSORS

(71) Applicant: Apple Inc., Cupertino, CA (US)

(72) Inventors: Miaolei YAN, Santa Clara, CA (US); Roberto M. RIBEIRO, San Jose, CA (US)

(21) Appl. No.: 16/041,655

(22) Filed: Jul. 20, 2018

Related U.S. Application Data
(60) Provisional application No. 62/535,788, filed on Jul. 21, 2017.

(51) Int. Cl. G01N 33/00

(52) U.S. Cl. CPC G01N 33/0016; G01N 33/0027; G01N 33/0073; G01N 33/007

(57) ABSTRACT

A miniature gas sensing device includes a silicon-based substrate embedded with multiple first heating elements. A number of electrodes are disposed on the silicon-based substrate. A gas-sensing layer covers the electrodes. A porous or mesoporous adsorbent layer selectively filters components of a gas mixture other than a target gas and allows the target gas to reach the gas-sensing layer. The first heating elements are operable to periodically regenerate sensing capabilities of at least the gas-sensing layer.

This application claims the benefit of priority under 35 U.S.C. § 119 from U.S. Provisional Patent Application 62/535,788 filed Jul. 21, 2017, which is incorporated herein by reference in its entirety.

### TECHNICAL FIELD

The present description relates generally to sensors, and more particularly, to chemically robust miniature gas sensors.

### BACKGROUND

Miniature gas sensors for consumer electronics represent a technology category that could enable upcoming features and/or products in applications such as, e.g., environmental and health monitoring, smart homes, and internet of things (IoT). Metal oxide (MOX) gas sensors are among the most promising technologies to be integrated with consumer electronic devices due to their small size, low power consumption, compatibility with semiconductor fabrication processes, and relatively simple architecture. Chemical poisoning and deactivation of the sensor materials in metal oxide sensors, however, can cause drift in both baseline resistance and sensitivity. This can lead to grossly inaccurate sensing performance and even premature device failures. Such drift and failure modes vary based on the operation and ambient environment; making global predictive software compensation to drift rather difficult. Various chemical species from the environment including siloxanes, sulfates, chlorides and phosphates have been identified as high-risk poisons. In addition, humidity (e.g., water vapor) can be a major interfering species that can reduce metal oxide sensor accuracy. Chemical poisoning and sensor drift pose great challenges to the mass market adoption of miniature gas sensors.

Producing a chemically robust miniature gas sensor that is resistant to chemical poisoning and interfering species enhances the long-term stability and lifetime of the sensor and would facilitate the mass market adoption of miniature gas sensors.

### BRIEF DESCRIPTION OF THE DRAWINGS

Certain features of the subject technology are set forth in the appended claims. However, for purposes of explanation, several embodiments of the subject technology are set forth in the following figures.

FIG. 1 is a schematic diagram illustrating an example of a miniature gas sensing device, in accordance with one or more aspects of the subject technology.

FIG. 2 is a schematic diagram illustrating an example of a miniature gas sensing device encased in an enclosure, in accordance with one or more aspects of the subject technology.

FIGS. 3A-3B are a top view and a cross-sectional schematic diagram illustrating an example of a miniature gas sensing device encased in an enclosure, in accordance with one or more aspects of the subject technology.

FIG. 4 is a flow diagram illustrating a method of operation of the miniature gas sensing devices of FIGS. 1 and 3B, in accordance with one or more aspects of the subject technology.

FIG. 5 is a flow diagram illustrating a method of operation of the miniature gas sensing device of FIG. 2, in accordance with one or more aspects of the subject technology.

FIG. 6 is a block diagram illustrating an example wireless communication device, within which one or more miniature gas sensors of the subject technology can be integrated.

### DETAILED DESCRIPTION

The detailed description set forth below is intended as a description of various configurations of the subject technology and is not intended to represent the only configurations in which the subject technology may be practiced. The appended drawings are incorporated herein and constitute a part of the detailed description. The detailed description includes specific details for the purpose of providing a thorough understanding of the subject technology. However, the subject technology is not limited to the specific details set forth herein and may be practiced without one or more of the specific details. In some instances, structures and components are shown in block diagram form in order to avoid obscuring the concepts of the subject technology.

In one or more aspects of the subject technology, solutions for producing robust miniature gas sensors are provided. The gas sensors of the subject technology include advantageous features such as improved stability, enhanced poisoning resistance, and reduced signal interference. The disclosed solutions can be employed for long-term implementation of environmental and health sensing and hazardous gas species detection in applications such as smart homes, internet of things (IoT), and other applications. The subject technology enables removing chemically poisonous and interfering species from the incoming gas stream, enhancing the sensor's poison resistance and stability and resulting in reduced signal drift over time.

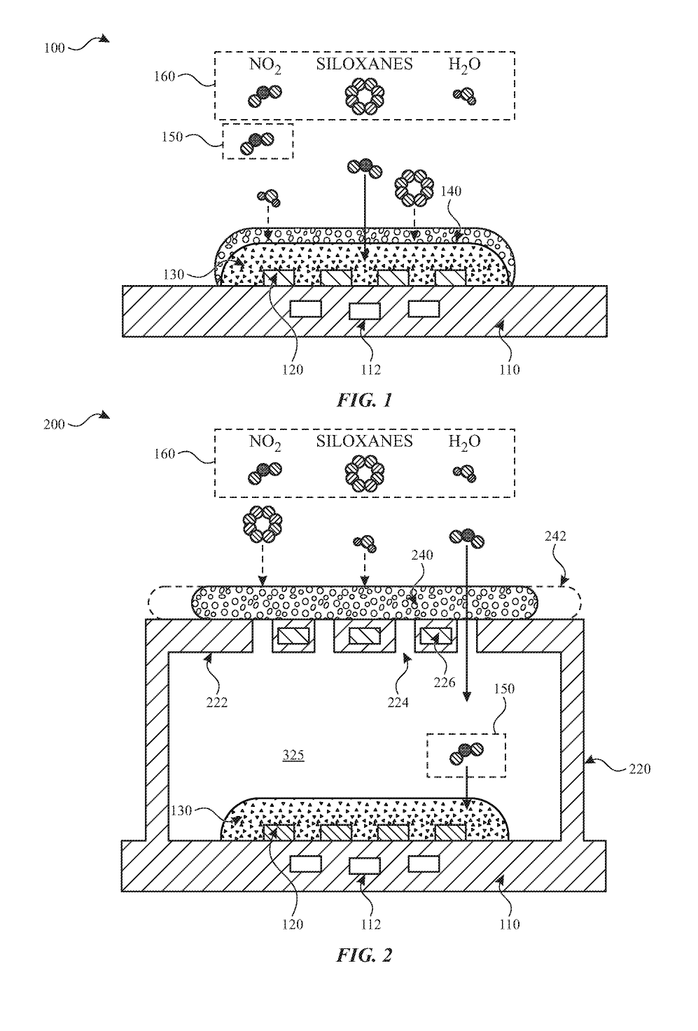

FIG. 1 is a schematic diagram illustrating an example of a miniature gas sensing device 100, in accordance with one or more aspects of the subject technology. The miniature gas sensing device 100 (hereinafter “gas sensor 100”) includes a substrate 110, a number of heating elements 112 (also referred to as the first heating elements), multiple electrodes 120, a gas-sensing layer 130 and a mesoporous adsorbent layer 140. The gas sensor 100 is capable of sensing a number of gases referred to as target gases 150. In some implementations, the gas sensor 100 is capable of sensing target gases 150 such as ozone (O₃), nitrogen dioxide (NO₂), nitrogen monoxide (NO), sulfur dioxide (SO₂), carbon monoxide (CO), methane (CH₄), carbon dioxide (CO₂) and volatile organic compounds (VOCs). In other implementations, the gas sensor 100 may be configured to sense other target gases. One of the advantages of the gas sensor 100 is that it can selectively filter components of a gas mixture other than a target gas 150, referred to as undesired species 160 and to allow the target gas 150 to reach the gas-sensing layer 130. The undesired species 160 may include any poisoning species such as, for example, siloxanes, sulfates, phosphates and chlorides, and/or interfering species such as water vapor. These undesired species, if not filtered (e.g., removed) by the gas sensor 100, may paralyze or adversely interfere with operation of the sensor 100. For example, the undesired species 160 may cause erroneous measurements or otherwise reduce the accuracy of the sensor 100.

In some implementations, the substrate 110 is a silicon-based substrate also referred to as a silicon-based membrane, although other membrane material suitable for integration into a particular application may be used. The substrate 110 may include the heating elements 112 as embedded elements such as micro electromechanical system (MEMS) hotplates. In some implementations, the heating elements 112 can include titanium nitride, which is compatible with complementary metal-oxide semiconductor (CMOS) process and has a high melting point (e.g., 2950 deg C.), although other suitable metals may be used. The heating elements 112 can be independently controlled (e.g., by a microcontroller or a general processor) and can be used to regulate the temperature of the gas-sensing layer 130. In some aspects, the microcontroller or the general processor can be in a host device such as smart phone or a smart watch, within which the gas sensor 100 in integrated. In some aspects, the heating elements 112 can be used to regenerate the sensing capabilities of the gas-sensing layer 130.

In some implementations, the electrodes 120 can be made of metals such as copper (Cu), aluminum (Al), silver (Ag), graphite (C), titanium (Ti), gold (Au), or other suitable metals, alloys or compounds. The electrodes 120 may be plated on the substrate in the form of a number of strips, for example, with suitable dimensions and intervening distances.

In some implementations, the gas-sensing layer 130 is made of a metal oxide, for example, a granular metal oxide semiconductor material including tin dioxide (SnO₂), tungsten trioxide (WO₃), indium oxide (In₂O₃), zinc oxide (ZnO2) or any binary combination of these or other materials. The gas-sensing layer 130 may detect the target gas 150 and convert the concentration of the gas target 150 into an electrical resistance. The gas-sensing layer 130 is formed on the electrodes 120, which are capable of sensing the electrical resistance that is proportional to the target gas concentration.

The adsorbent layer 140 (hereinafter “adsorbent layer 140”) is disposed over the gas-sensing layer 130 and substantially covers the gas-sensing layer 130. In some implementations, the adsorbent layer 140 includes mesoporous silica, silica gel, activated silica, zeolite, metal organic framework or other suitable material. In one or more implementations, the adsorbent layer 140 may be a thin layer with a thickness of hundreds of nanometers to a few microns, for example, within a range of about 0.2-3 μm. In other implementations, the thickness of the adsorbent layer 140 can be within a range of about 0.1-5 μm. The adsorbent layer 140 can selectively filter (e.g., remove) undesired components (e.g., species) of a gas mixture other than a target gas 150 and include pores that allow the target gas 150 to reach the gas-sensing layer 130. The undesired species 160 may include any poisoning species such as, for example, siloxanes, sulfates, phosphates and chlorides, and/or interfering species such as water vapor. The undesired species 160 can be adsorbed by the adsorbent layer 140 and can be later released from the adsorbent layer 140 via a regeneration operation discussed in more details herein. In some implementations, the adsorbent layer 140 may include a porous or mesoporous adsorbent material that can adsorb the undesired components (e.g., species) of the gas mixture other than a target gas 150 and later on release the undesired components in response to the regeneration operation.

FIG. 2 is a schematic diagram illustrating an example of a miniature gas sensing device 200 encased in an enclosure 220, in accordance with one or more aspects of the subject technology. The miniature gas sensing device 200 (hereinafter “gas sensor 200”) includes the substrate 110, the first heating elements 112, the electrodes 120, the gas-sensing layer 130, the enclosure 220, and a porous/mesoporous adsorbent layer 240. The description of the structures and operations of substrate 110, the first heating elements 112, the electrodes 120 and the gas-sensing layer 130 are as described above with respect to the gas sensor 100 of FIG. 1 and are skipped here for brevity. The structure and operation of the adsorbent layer 240 is similar to the structure and operation of the adsorbent layer 140 as described above with respect to FIG. 1.

The enclosure 220 can be made of the same material as the substrate 110 (e.g., a silicon-based material) or any other suitable material. The enclosure 220 has a top side 222 parallel to a plane of the substrate 110 and sidewalls attached to the substrate 110. In some implementations, the substrate 110 and the enclosure 220 can be built of the silicon-based material and by using the manufacturing techniques of integrated circuits (ICs) such as patterning and etching and bonded together. In some aspects the enclosure 220 includes a number of holes (openings) 224 in the top side 222. The adsorbent layer 240 is formed over a top (external) surface of the top side 222 of the enclosure 220.

The adsorbent layer 240 may, for example, be formed by liquid phase deposition and by using a sacrificial layer 242 that can provide structural support for the liquid phase adsorbent deposition. Examples of the sacrificial layer 242 include filtering paper, cotton, or Gore-Tex membrane (e.g., made of polytetrafluoroethylene). The adsorbent layer 240 is capable of adsorbing the undesired species 160 and allows the target gas 150 to enter the enclosure through the holes 224. In some implementations, the enclosure 220 is capable of thermally isolating the gas content of the enclosure from the outside air and allows the temperature inside the enclosure to rise to a suitable temperature. In some implementations, the enclosure 220 includes second heating elements 226 (e.g., MEMS hotplates) embedded in the top side 222 of the enclosure 220. The structure and operation of the second heating elements 226 can be similar to the first heating elements 112. The second heating elements 226 can be used for regulating the temperature of and/or regeneration of the adsorbent layer 240, as described below.

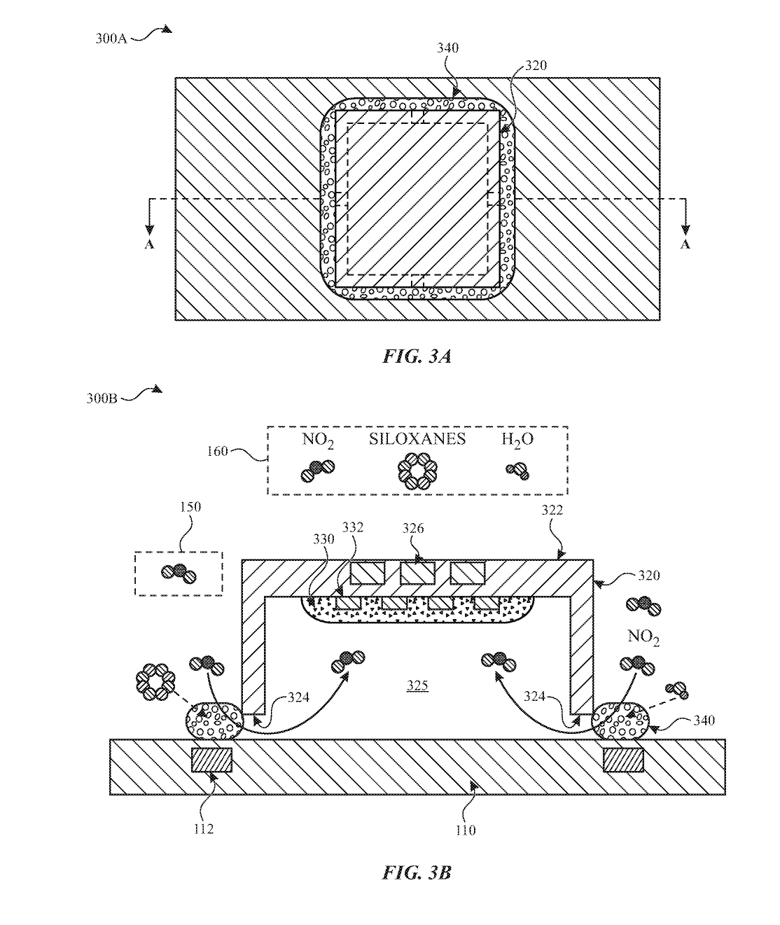

FIGS. 3A-3B are a top view 300A and a schematic diagram illustrating an example of a miniature gas sensing device 300B encased in an enclosure 320, in accordance with one or more aspects of the subject technology. The top view 300A shown in FIG. 3A corresponds to the miniature gas sensing device 300B (hereinafter “gas sensor 300B”) shown in FIG. 3B. The top view 300A includes the enclosure 320 and the adsorbent layer 340 that will be described below with respect to FIG. 3B.

The schematic diagram of the gas sensor 300B, as shown in the FIG. 3B, is a cross-sectional view across AA′ of FIG. 3A. The gas sensor 300B includes the substrate 110, the first heating elements 112, the enclosure 320, electrodes 332, a gas-sensing layer 330, and a adsorbent layer 340 (hereinafter “adsorbent layer 340”). The description of the structures and operations of the substrate 110 and the first heating elements 112 are as described above with respect to the gas sensor 100 of FIG. 1 and are skipped here for brevity. The structure and operation of the adsorbent layer 340 is similar to the structure and operation of the adsorbent layer 140 as described above with respect to FIG. 1, except that the adsorbent layer 340 is formed around the junction (interface) of the sidewalls of the enclosure 320 with the substrate 110. In some implementations, the adsorbent layer 340 is disposed over one or more openings 324 in on one or more sidewalls of the enclosure at the junction of the sidewalls with the substrate 110.

The structure and operation of the gas-sensing layer 330 and the electrodes 332 are similar to the structure and operation of the adsorbent layer 140 as described above with respect to FIG. 1, except that in the gas sensor 300B, the gas-sensing layer 330 and the electrodes 332 are built over a first surface (internal surface) of a first side (e.g., top side) 322 of the enclosure 320 that faces the substrate 110.

In some implementations, the enclosure 320 is MEMS structure including a silicon lid. The enclosure 320 includes sidewalls that are attached to the substrate 110. In some implementations, the MEMS structure is integrated with electrodes (e.g., interdigitated fingers) 332, the sensing layer 330 is formed (e.g., deposited) on the first surface of the top side 322 of the enclosure 320, and lateral notches (e.g., openings 324) are made on the sidewalls to serve as gas exchange (e.g., through the adsorbent layer 340). The MEMS structure is then bonded (e.g., wafer bonded) to the substrate 110. The enclosure 320 may provide a thermal isolation of the gas content of the enclosure 320 from the outside air. The enclosure 320 may also protect the gas-sensing layer 330 from the outside world, and any gas reaching the sensor has to pass through the adsorbent layer 340. In the implementations discussed above, the enclosure 320 can maintain a high temperature sufficient for regeneration of sensing capabilities of the gas sensor layer 330 when the first heating elements 112 are on. In some implementations, the first (top) side 322 of the enclosure 320 can have embedded second heating elements 326, which are functionally and structurally similar to the first heating elements 112, as discussed above with respect to FIG. 1, and can be used for regulating the temperature of the gas-sensing layer 330.

In some aspects, the first heating elements 112 and the second heating elements 326 can be used to perform simultaneous adsorption and sensing operations and regeneration of adsorption and sensing capabilities of the gas sensor 300B as described in more details below. In one or more implementations, the first heating elements 112 and the second heating elements 326 can be simultaneously or independently controlled, for example, by a microcontroller or a processor, for example, of a host device such as smart phone or a smart watch.

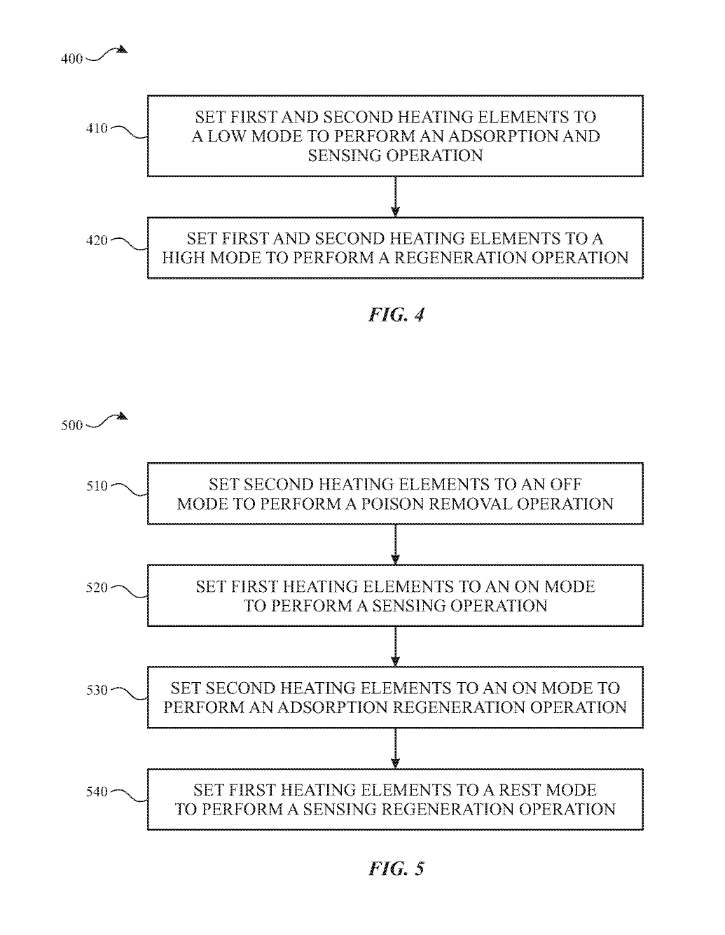

FIG. 4 is a flow diagram illustrating a method 400 of operation of the miniature gas sensing devices 100 and 300B of FIGS. 1 and 3B, in accordance with one or more aspects of the subject technology. The method 400 includes operation 410 and 420. In the operation 410, the first heating elements 112 of FIGS. 1 and 3B and the second heater elements 326 of FIG. 3B (if exist) are set to a low mode to perform an adsorption and sensing operation. In the adsorption operation, the adsorbent layer 140 of FIG. 1 or the adsorbent layer 340 of FIG. 3B removes undesired species (e.g., 160 of FIG. 1 or FIG. 3B) from the gas mixture and the gas-sensing layers 130 of FIG. 1 or 330 of FIG. 3B are ready to sense the target gas 150 of FIG. 1 or FIG. 3B and convert the concentration of the target gas to a resistance signal.

In the operation 420, the first heating elements 112 and the second heating elements 326 (when present) are set to a high mode to perform the regeneration operations. In the regeneration operations, the adsorption capabilities of the adsorbent layers 140 and 340 and the sensing capabilities of the gas sensor layers 130 and 330 are regenerated by the high temperature (e.g., a few hundred deg. C., for example, within a range of about 100 deg. C. to 600 deg. C.).

FIG. 5 is a flow diagram illustrating a method 500 of operation of the miniature gas sensing device 200 of FIG. 2, in accordance with one or more aspects of the subject technology. The method 500 includes operation 510, 520, 530 and 540. In the operation 510, the second heating elements 226 of FIG. 2 are set to an off mode to perform a poison removal operation. In the poison removal operation, the adsorbent layer 240 of FIG. 2 filters the undesired species 160 as described above with respect to FIG. 1. In the operation 520, the first heating elements 112 are set to an on mode (e.g., a high temperature of a few hundred deg. C.) to perform a sensing operation. In the sensing operation, the gas-sensing layer 130 of FIG. 2 convert the target gas 150 of FIG. 2 to resistance values (an electrical signal) as described above with respect to FIG. 1.

In the operation 530, the second heating elements 226 are set to an on mode to perform an adsorption regeneration operation. In the adsorption regeneration operation, the undesired species 160 adsorbed by the adsorbent layer 240 are decomposed and adsorption capabilities of the adsorbent layer 240 are regenerated as described above with respect to FIG. 1.

In the operation 540, the first heating elements 112 are set to a rest mode (e.g., low temperature such as room temperature) to perform a sensing regeneration operation. In the sensing regeneration operation, gases adsorbed by the gas-sensing layer 130 of FIG. 2 are desorbed and decomposed and the sensing capabilities of the gas-sensing layer 130 are regenerated.

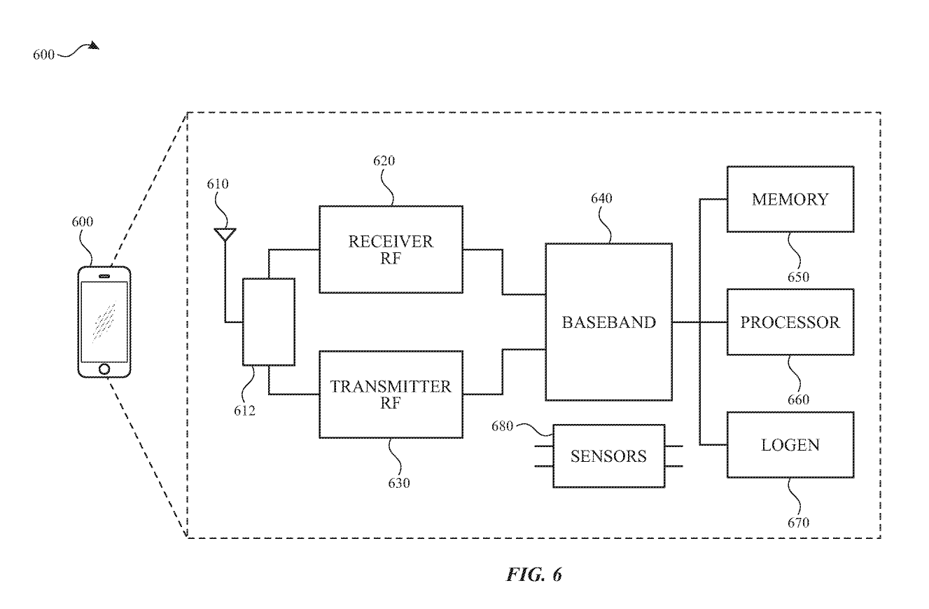

FIG. 6 is a block diagram illustrating an example wireless communication device, in which one or more miniature gas sensors of the subject technology can be implemented. The wireless communication device 600 may comprise a radio-frequency (RF) antenna 610, a receiver 620, a transmitter 630, a baseband processing module 640, a memory 650, a processor 660, a local oscillator generator (LOGEN) 670 and one or more sensors 680. In various embodiments of the subject technology, one or more of the blocks represented in FIG. 6 may be integrated on one or more semiconductor substrates. For example, the blocks 620-670 may be realized in a single chip or a single system on a chip, or may be realized in a multi-chip chipset.

The receiver 620 may comprise suitable logic circuitry and/or code that may be operable to receive and process signals from the RF antenna 610. The receiver 620 may, for example, be operable to amplify and/or down-convert received wireless signals. In various embodiments of the subject technology, the receiver 620 may be operable to cancel noise in received signals and may be linear over a wide range of frequencies. In this manner, the receiver 620 may be suitable for receiving signals in accordance with a variety of wireless standards, Wi-Fi, WiMAX, Bluetooth, and various cellular standards. In various embodiments of the subject technology, the receiver 620 may not require any SAW filters and few or no off-chip discrete components such as large capacitors and inductors.

The transmitter 630 may comprise suitable logic circuitry and/or code that may be operable to process and transmit signals from the RF antenna 610. The transmitter 630 may, for example, be operable to up-convert baseband signals to RF signals and amplify RF signals. In various embodiments of the subject technology, the transmitter 630 may be operable to up-convert and amplify baseband signals processed in accordance with a variety of wireless standards. Examples of such standards may include Wi-Fi, WiMAX, Bluetooth, and various cellular standards. In various embodiments of the subject technology, the transmitter 630 may be operable to provide signals for further amplification by one or more power amplifiers.

The duplexer 612 may provide isolation in the transmit band to avoid saturation of the receiver 620 or damaging parts of the receiver 620, and to relax one or more design requirements of the receiver 620. Furthermore, the duplexer 612 may attenuate the noise in the receive band. The duplexer may be operable in multiple frequency bands of various wireless standards.

The baseband processing module 640 may comprise suitable logic, circuitry, interfaces, and/or code that may be operable to perform processing of baseband signals. The baseband processing module 640 may, for example, analyze received signals and generate control and/or feedback signals for configuring various components of the wireless communication device 600, such as the receiver 620. The baseband processing module 640 may be operable to encode, decode, transcode, modulate, demodulate, encrypt, decrypt, scramble, descramble, and/or otherwise process data in accordance with one or more wireless standards.

The processor 660 may comprise suitable logic, circuitry, and/or code that may enable processing data and/or controlling operations of the wireless communication device 600. In this regard, the processor 660 may be enabled to provide control signals to various other portions of the wireless communication device 600. The processor 660 may also control transfers of data between various portions of the wireless communication device 600. Additionally, the processor 660 may enable implementation of an operating system or otherwise execute code to manage operations of the wireless communication device 600. In some aspects, the processor 660 may partially or entirely perform operations described in the methods 400 and 500 of FIGS. 4 and 5, for example, by controlling the heating elements (e.g., 112 of FIG. 1 and/or 226 of FIG. 2).

The memory 650 may comprise suitable logic, circuitry, and/or code that may enable storage of various types of information such as received data, generated data, code, and/or configuration information. The memory 650 may comprise, for example, RAM, ROM, flash, and/or magnetic storage. In various embodiment of the subject technology, information stored in the memory 650 may be utilized for configuring the receiver 620 and/or the baseband processing module 640. In some embodiments, the memory 650 may store resistance values as sensed, for example, by the electrodes 120 attached to the gas sensor 130 of FIGS. 1 and 2.

The local oscillator generator (LOGEN) 670 may comprise suitable logic, circuitry, interfaces, and/or code that may be operable to generate one or more oscillating signals of one or more frequencies. The LOGEN 670 may be operable to generate digital and/or analog signals. In this manner, the LOGEN 670 may be operable to generate one or more clock signals and/or sinusoidal signals. Characteristics of the oscillating signals such as the frequency and duty cycle may be determined based on one or more control signals from, for example, the processor 660 and/or the baseband processing module 640.

In operation, the processor 660 may configure the various components of the wireless communication device 600 based on a wireless standard according to which it is desired to receive signals. Wireless signals may be received via the RF antenna 610 and amplified and down-converted by the receiver 620. The baseband processing module 640 may perform noise estimation and/or noise cancellation, decoding, and/or demodulation of the baseband signals. In this manner, information in the received signal may be recovered and utilized appropriately. For example, the information may be audio and/or video to be presented to a user of the wireless communication device, data to be stored to the memory 650, and/or information affecting and/or enabling operation of the wireless communication device 600. The baseband processing module 640 may modulate, encode, and perform other processing on audio, video, and/or control signals to be transmitted by the transmitter 630 in accordance with various wireless standards.

The one or more sensors 680 may include the miniature gas sensors of the subject technology as shown in FIGS. 1, 2, 3A and 3B and described above. The miniature gas sensors of the subject technology can be readily integrated into the communication device 600, in particular when the communication device 600 is a smart mobile phone or a smart watch.

The previous description is provided to enable any person skilled in the art to practice the various aspects described herein. Various modifications to these aspects will be readily apparent to those skilled in the art, and the generic principles defined herein may be applied to other aspects. Thus, the claims are not intended to be limited to the aspects shown herein, but are to be accorded the full scope consistent with the language claims, wherein reference to an element in the singular is not intended to mean “one and only one” unless specifically so stated, but rather “one or more.” Unless specifically stated otherwise, the term “some” refers to one or more. Pronouns in the masculine (e.g., his) include the feminine and neuter gender (e.g., her and its) and vice versa. Headings and subheadings, if any, are used for convenience only and do not limit the subject disclosure.

The predicate words “configured to”, “operable to”, and “programmed to” do not imply any particular tangible or intangible modification of a subject, but, rather, are intended to be used interchangeably. For example, a processor configured to monitor and control an operation or a component may also mean the processor being programmed to monitor and control the operation or the processor being operable to monitor and control the operation. Likewise, a processor configured to execute code can be construed as a processor programmed to execute code or operable to execute code.

A phrase such as an “aspect” does not imply that such aspect is essential to the subject technology or that such aspect applies to all configurations of the subject technology. A disclosure relating to an aspect may apply to all configurations, or one or more configurations. A phrase such as an aspect may refer to one or more aspects and vice versa. A phrase such as a “configuration” does not imply that such configuration is essential to the subject technology or that such configuration applies to all configurations of the subject technology. A disclosure relating to a configuration may apply to all configurations, or one or more configurations. A phrase such as a configuration may refer to one or more configurations and vice versa.

The word “example” is used herein to mean “serving as an example or illustration.” Any aspect or design described herein as “example” is not necessarily to be construed as preferred or advantageous over other aspects or designs.

All structural and functional equivalents to the elements of the various aspects described throughout this disclosure that are known or later come to be known to those of ordinary skill in the art are expressly incorporated herein by reference and are intended to be encompassed by the claims. Moreover, nothing disclosed herein is intended to be dedicated to the public regardless of whether such disclosure is explicitly recited in the claims. No claim element is to be construed under the provisions of 35 U.S.C. § 112, sixth paragraph, unless the element is expressly recited using the phrase “means for” or, in the case of a method claim, the element is recited using the phrase “step for.” Furthermore, to the extent that the term “include,” “have,” or the like is used in the description or the claims, such term is intended to be inclusive in a manner similar to the term “comprise” as “comprise” is interpreted when employed as a transitional word in a claim.

What is claimed is:

1. A miniature gas sensing device, the device comprising:
a silicon-based substrate embedded with a plurality of first heating elements;
a plurality of electrodes disposed on the silicon-based substrate;
a gas-sensing layer covering the plurality of electrodes; and
an adsorbent layer configured to selectively filter components of a gas mixture other than a target gas and to allow the target gas to reach the gas-sensing layer,
wherein the first heating elements are operable to periodically regenerate sensing capabilities of at least the gas-sensing layer.

2. The device of claim 1, wherein the adsorbent layer comprises a porous or mesoporous layer and is disposed over the gas-sensing layer and substantially covers the gas-sensing layer.

3. The device of claim 1, wherein the first heating elements are operable to periodically regenerate sensing capabilities of the gas-sensing layer and adsorption capabilities of the adsorbent layer.

4. The device of claim 1, wherein the first heating elements are operable in a low mode that allows simultaneous adsorption of the component of the gas mixture other than the target gas and converting target gas signals to resistance values.

5. The device of claim 1, wherein the first heating elements are operable in a high mode that allows simultaneous regeneration of sensing capabilities of the gas-sensing layer and adsorption capabilities of the adsorbent layer.

6. The device of claim 1, wherein the adsorbent layer comprises at least one of mesoporous silica, silica gel, activated silica, zeolite or metal organic framework.

7. The device of claim 1, wherein the adsorbent layer has a thickness within a range of about 0.2-3 μm.

8. The device of claim 1, wherein the gas-sensing layer comprises a granular metal oxide semiconductor material including at least one of tin dioxide (SnO₂), tungsten trioxide (WO₃), indium oxide (In₂O₃), zinc oxide (ZnO) and is configured to convert a target gas concentration to an electrical resistance.

9. The device of claim 1, wherein the target gas comprises at least one of ozone (O₃), nitrogen dioxide (NO₂), nitrogen monoxide (NO), sulfur dioxide (SO₂), carbon monoxide (CO), methane (CH₄), and volatile organic compounds (VOCs), and wherein the components of a gas mixture other than the target gas comprises poisoning species including siloxanes, sulfates, phosphates and chlorides, and/or interfering species such as water vapor.

10. The device of claim 1, wherein the adsorbent layer is disposed over an enclosure above the gas-sensing layer, wherein the enclosure includes at least one embedded second heating element and at least one opening to allow the target gas to reach the gas-sensing layer.

11. The device of claim 10, wherein the enclosure includes a plurality of second heating elements operable to periodically regenerate adsorption capabilities of the layer.

12. The device of claim 1, wherein the adsorbent layer is disposed over the silicon-based substrate and at one or more openings of an enclosure, wherein the enclosure is disposed over the silicon-based substrate and the one or more openings of the enclosure are made at an interface of the enclosure with the silicon-based substrate.

13. The device of claim 12, wherein the plurality of electrodes and the gas-sensing layer are disposed over an internal surface of the enclosure in parallel with and facing the silicon-based substrate, and wherein the enclosure includes one or more second heating elements embedded in a side of the enclosure in parallel with the silicon-based substrate.

14. A miniature gas sensing device, the device comprising:
a substrate embedded with one or more first heating elements;
a plurality of electrodes disposed on the substrate;
a gas-sensing layer covering the plurality of electrodes;
an enclosure disposed over the substrate, the enclosure including one or more openings in a first side of the enclosure; and
an adsorbent layer configured to selectively filter components of a gas mixture other than a target gas and to allow the target gas to reach the gas-sensing layer through the one or more openings in the first side of the enclosure,
wherein the first side of the enclosure is in parallel with the substrate.

15. The device of claim 14, wherein the first side of the enclosure further includes one or more second independently operable heating elements, wherein the one or more first heating elements are operable to periodically regenerate sensing capabilities of the gas-sensing layer and the one or more second heating elements are operable to periodically regenerate adsorption capabilities of the adsorbent layer, and wherein the adsorbent layer comprises a porous or a mesoporous layer.

16. The device of claim 15, wherein the one or more first heating elements are operable to be in an on mode and the second heating elements are operable to be in an off mode to allow a poison removal and sensing operation.

17. The device of claim 15, wherein the one or more first heating elements are operable to be in a reset mode and the second heating elements are operable to be in an on mode to allow a regeneration operation.

18. The device of claim 14, wherein the substrate comprises a silicon-based substrate, wherein the adsorbent layer comprises at least one of mesoporous silica, silica gel, activated silica, zeolite or metal organic framework, and wherein the adsorbent layer has a thickness within a range of about 0.1-5 μm.

19. A system comprising:
a communication device; and
a miniature gas sensor integrated within the communication device, the gas sensor comprising:
a substrate embedded with one or more first heating elements;
an enclosure disposed over the substrate, the enclosure including a first side in parallel with a plane of the substrate;
a plurality of electrodes disposed on an internal surface of the first side of the enclosure facing the substrate;
a gas-sensing layer covering the plurality of electrodes; and
an adsorbent layer configured to selectively filter components of a gas mixture other than a target gas and to allow the target gas to reach the gas-sensing layer,
wherein the adsorbent layer is disposed over one or more openings in on one or more sidewalls of the enclosure at an interface of the one or more sidewalls with the substrate.

20. The system of claim 19, wherein the communication device comprises a handheld communication device including a smart phone or a smart watch, wherein the first side of the enclosure includes a plurality of second heating elements, wherein the first and second heating elements are operable to be in a low mode to allow an adsorption and sensing operation, wherein the first and second heating elements are operable to be in a high mode to allow a regeneration operation, wherein the substrate comprises a silicon-based substrate, wherein the adsorbent layer comprises a porous or a mesoporous layer, and wherein the adsorbent layer comprises at least one of mesoporous silica, silica gel or activated silica, zeolite or metal organic framework, and wherein the adsorbent layer has a thickness within a range of about 0.1-5 μm.
